(19) United States
(12) Patent Application Publication
(10) Pub. No.: US 2007/0112000 A1
(43) Pub. Date: May 17, 2007

(54) Chemical compounds

(75) Inventors: Peter John Barton, Cheshire (GB); Roger John Butlin, Cheshire (GB); Janet Elizabeth Pease, Cheshire (GB)

Correspondence Address: MORGAN LEWIS & BOCKIUS LLP, 1111 PENNSYLVANIA AVENUE NW, WASHINGTON, DC, 20004, US

(73) Assignee: AstraZeneca R&D Alderley, Cheshire (GB)

(21) Appl. No.: 10/578,444

(22) PCT Filed: Nov. 4, 2004
(86) PCT No.: PCT/GB04/04650

§ 371 Date: May 5, 2006

(30) Foreign Application Priority Data
Nov. 7, 2003 (GB) 0326029.6

(51) Int. Cl. A61K 31/5377; A61K 31/4709; A61K 31/4545; A61K 31/454; A61K 31/445; C07D 413/02; C07D 409/02

(52) U.S. Cl. 514/235.2; 514/317; 514/320; 514/326; 514/314; 514/318; 544/130; 546/169; 546/194; 546/196; 546/208; 546/221

(57) ABSTRACT

The use of compounds of formula (I) wherein variable groups are defined within; in the manufacture of medicaments for use in the inhibition of 11βHSD1, process for making them, certain compounds within the definition of the formula (I) and pharmaceutical compositions comprising them are described. The compounds are useful in the treatment of metabolic syndrome, diabetes and obesity.

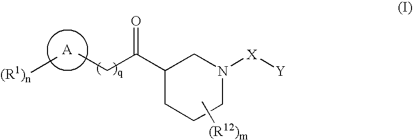

This invention relates to chemical compounds, or pharmaceutically acceptable salts thereof. These compounds possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

Glucocorticoids (cortisol in man, corticosterone in rodents) are counter regulatory hormones i.e. they oppose the actions of insulin (Dallman M F, Strack A M, Akana S F et al. 1993; Front Neuroendocrinol 14, 303-347). They regulate the expression of hepatic enzymes involved in gluconeogenesis and increase substrate supply by releasing glycerol from adipose tissue (increased lipolysis) and amino acids from muscle (decreased protein synthesis and increased protein degradation). Glucocorticoids are also important in the differentiation of pre-adipocytes into mature adipocytes which are able to store triglycerides (Bujalska U et al. 1999; Endocrinology 140, 3188-3196). This may be critical in disease states where glucocorticoids induced by “stress” are associated with central obesity which itself is a strong risk factor for type 2 diabetes, hypertension and cardiovascular disease (Bjorntorp P & Rosmond R 2000; Int. J. Obesity 24, S80-S85)

It is now well established that glucocorticoid activity is controlled not simply by secretion of cortisol but also at the tissue level by intracellular interconversion of active cortisol and inactive cortisone by the 11-beta hydroxysteroid dehydrogenases, 11βHSD1 (which activates cortisone) and 11βHSD2 (which inactivates cortisol) (Sandeep T C & Walker B R 2001 Trends in Endocrinol & Metab. 12, 446-453). That this mechanism may be important in man was initially shown using carbenoxolone (an anti-ulcer drug which inhibits both 11βHSD1 and 2) treatment which (Walker B R et al. 1995; J. Clin. Endocrinol. Metab. 80, 3155-3159) leads to increased insulin sensitivity indicating that 11βHSD1 may well be regulating the effects of insulin by decreasing tissue levels of active glucocorticoids (Walker B R et al. 1995; J. Clin. Endocrinol. Metab. 80, 3155-3159).

Clinically, Cushing's syndrome is associated with cortisol excess which in turn is associated with glucose intolerance, central obesity (caused by stimulation of pre-adipocyte differentiation in this depot), dyslipidaemia and hypertension. Cushing's syndrome shows a number of clear parallels with metabolic syndrome. Even though the metabolic syndrome is not generally associated with excess circulating cortisol levels (Jessop D S et al. 2001; J. Clin. Endocrinol. Metab. 86, 4109-4114) abnormally high 11βHSD1 activity within tissues would be expected to have the same effect. In obese men it was shown that despite having similar or lower plasma cortisol levels than lean controls, 11βHSD1 activity in subcutaneous fat was greatly enhanced (Rask E et al. 2001; J. Clin. Endocrinol. Metab. 1418-1421). Furthermore, the central fat, associated with the metabolic syndrome expresses much higher levels of 11βHSD1 activity than subcutaneous fat (Bujalska I J et al. 1997; Lancet 349, 1210-1213). Thus there appears to be a link between glucocorticoids, 11βHSD1 and the metabolic syndrome.

11βHSD1 knock-out mice show attenuated glucocorticoid-induced activation of gluconeogenic enzymes in response to fasting and lower plasma glucose levels in response to stress or obesity (Kotelevtsev Y et al. 1997; Proc. Natl. Acad. Sci USA 94, 14924-14929) indicating the utility of inhibition of 11βHSD1 in lowering of plasma glucose and hepatic glucose output in type 2 diabetes. Furthermore, these mice express an anti-atherogenic lipoprotein profile, having low triglycerides, increased HDL cholesterol and increased apo-lipoprotein AI levels. (Morton N M et al. 2001; J. Biol. Chem. 276, 41293-41300). This phenotype is due to an increased hepatic expression of enzymes of fat catabolism and PPARα. Again this indicates the utility of 11βHSD1 inhibition in treatment of the dyslipidaemia of the metabolic syndrome.

The most convincing demonstration of a link between the metabolic syndrome and 11βHSD1 comes from recent studies of transgenic mice over-expressing 11βHSD1 (Masuzaki H et al. 2001; Science 294, 2166-2170). When expressed under the control of an adipose specific promoter, 11βHSD1 transgenic mice have high adipose levels of corticosterone, central obesity, insulin resistant diabetes, hyperlipidaemia and hyperphagia. Most importantly, the increased levels of 11βHSD1 activity in the fat of these mice are similar to those seen in obese subjects. Hepatic 11βHSD1 activity and plasma corticosterone levels were normal, however, hepatic portal vein levels of corticosterone were increased 3 fold and it is thought that this is the cause of the metabolic effects in liver.

Overall it is now clear that the complete metabolic syndrome can be mimicked in mice simply by overexpressing 11βHSD1 in fat alone at levels similar to those in obese man.

11βHSD1 tissue distribution is widespread and overlapping with that of the glucocorticoid receptor. Thus, 11βHSD1 inhibition could potentially oppose the effects of glucocorticoids in a number of physiological/pathological roles. 11βHSD1 is present in human skeletal muscle and glucocorticoid opposition to the anabolic effects of insulin on protein turnover and glucose metabolism are well documented (Whorwood C B et al. 2001; J. Clin. Endocrinol. Metab. 86, 2296-2308). Skeletal muscle must therefore be an important target for 11βHSD1 based therapy.

Glucocorticoids also decrease insulin secretion and this could exacerbate the effects of glucocorticoid induced insulin resistance. Pancreatic islets express 11βHSD1 and carbenoxolone can inhibit the effects of 11-dehydocorticosterone on insulin release (Davani B et al. 2000; J. Biol. Chem. 275, 34841-34844). Thus in treatment of diabetes 11βHSD1 inhibitors may not only act at the tissue level on insulin resistance but also increase insulin secretion itself.

Skeletal development and bone function is also regulated by glucocorticoid action. 11βHSD1 is present in human bone osteoclasts and osteoblasts and treatment of healthy volunteers with carbenoxolone showed a decrease in bone resorption markers with no change in bone formation markers (Cooper M S et al 2000; Bone 27, 375-381). Inhibition of 11βHSD1 activity in bone could be used as a protective mechanism in treatment of osteoporosis.

Glucocorticoids may also be involved in diseases of the eye such as glaucoma. 11βHSD1 has been shown to affect intraocular pressure in man and inhibition of 11βHSD1 may be expected to alleviate the increased intraocular pressure associated with glaucoma (Rauz S et al. 2001; Investigative Opthalmology & Visual Science 42, 2037-2042).

There appears to be a convincing link between 11βHSD1 and the metabolic syndrome both in rodents and in humans. Evidence suggests that a drug which specifically inhibits 11βHSD1 in type 2 obese diabetic patients will lower blood glucose by reducing hepatic gluconeogenesis, reduce central obesity, improve the atherogenic lipoprotein phenotype, lower blood pressure and reduce insulin resistance. Insulin effects in muscle will be enhanced and insulin secretion from the beta cells of the islet may also be increased.

Currently there are two main recognised definitions of metabolic syndrome.

1) The Adult Treatment Panel (ATP III 2001 JMA) definition of metabolic syndrome indicates that it is present if the patient has three or more of the following symptoms:

Waist measuring at least 40 inches (102 cm) for men, 35 inches (88 cm) for women;
Serum triglyceride levels of at least 150 mg/dl (1.69 mmol/l);
HDL cholesterol levels of less than 40 mg/dl (1.04 mmol/l) in men, less than 50 mg/dl (1.29 mmol/l) in women;
Blood pressure of at least 135/80 mm Hg; and/or
Blood sugar (serum glucose) of at least 110 mg/dl (6.1 mmol/l).
   2) The WHO consultation has recommended the following definition which does not imply causal relationships and is suggested as a working definition to be improved upon in due course:
The patient has at least one of the following conditions: glucose intolerance, impaired glucose tolerance (IGT) or diabetes mellitus and/or insulin resistance; together with two or more of the following:
Raised Arterial Pressure;
Raised plasma triglycerides
Central Obesity
Microalbuminuria

We have found that the compounds defined in the present invention, or a pharmaceutically acceptable salt thereof, are effective 11βHSD1 inhibitors, and accordingly have value in the treatment of disease states associated with metabolic syndrome.

Accordingly there is provided the use of a compound of formula (I): 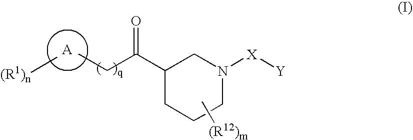
wherein:

Ring A is selected from carbocyclyl or heterocyclyl; wherein if said heterocyclyl contains an —NH— moiety that nitrogen may be optionally substituted by a group selected from R⁹;

R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄-alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;

n is 0-5; wherein the values of R¹ may be the same or different;

X is a direct bond, —C(O)—, —S(O)₂—, —C(O)NR¹¹—, —C(S)NR¹¹—, —C(O)O—, —C(═NR¹¹)— or —CH₂—; wherein R¹¹ is selected from hydrogen, C₁₋₄alkyl, carbocyclyl and heterocyclyl;

Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;

R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;

R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸; and wherein if said heterocyclyl contains an —NH— moiety that nitrogen may be optionally substituted by a group selected from R¹³;

R⁴, R⁵, R⁷, R⁹ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;

R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;

R¹² is hydroxy, methyl, ethyl, propyl or trifluoromethyl;

m is 0 or 1;

q is 0 or 1;

or a pharmaceutically acceptable salt thereof;

in the manufacture of a medicament for use in the inhibition of 11βHSD1.

For the avoidance of doubt, where X is —C(O)NR¹¹—, —C(S)NR¹¹— or —C(O)O— is it the C(O) or the C(S) that is attached to the nitrogen of the pyrrolidine ring in formula (I).

In another aspect, there is provided the use of a compound of formula (I): wherein:

Ring A is selected from carbocyclyl or heterocyclyl; wherein if said heterocyclyl contains an —NH— moiety that nitrogen may be optionally substituted by a group selected from R⁹;

R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄-alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;

n is 0-5; wherein the values of R¹ may be the same or different;

X is a direct bond, —C(O)—, —S(O)₂—, —C(O)NR¹¹—, —C(S)NR¹¹—, —C(O)O—, —C(═NR¹¹)— or —CH₂—; wherein R¹¹ is selected from hydrogen, C₁₋₄alkyl, carbocyclyl and heterocyclyl;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;

R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;

R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸; and wherein if said heterocyclyl contains an —NH— moiety that nitrogen may be optionally substituted by a group selected from R¹³;

R⁴, R⁵, R⁷, R⁹ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;

R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;

R¹² is hydroxy, methyl, ethyl or propyl;

m is 0 or 1;

q is 0 or 1;

or a pharmaceutically acceptable salt thereof;

in the manufacture of a medicament for use in the inhibition of 11βHSD1.

According to a further feature of the invention there is provided a compound of formula (IA′): 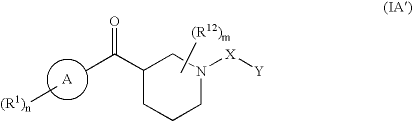
wherein:

Ring A is selected from phenyl, pyridyl, thienyl, furyl or thiazolyl;

R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl or heterocyclyl; wherein R¹ may be optionally substituted on carbon by one or more groups selected from R³; and wherein if said heterocyclyl contains an —NH— moiety that nitrogen may be optionally substituted by a group selected from R⁴;

n is 0-5; wherein the values of R¹ may be the same or different;

X is a —C(O)—, —S(O)₂—, —C(O)NR¹¹—, —C(S)NR¹¹—, —C(O)O— or —C(═NR¹¹)—; wherein R¹¹ is selected from hydrogen, C₁₋₄alkyl, carbocyclyl and heterocyclyl;

Y is C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;

R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl or heterocyclyl; wherein R² may be optionally substituted on carbon by one or more groups selected from R⁶; and wherein if said heterocyclyl contains an —NH— moiety that nitrogen may be optionally substituted by a group selected from R⁷;

R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄-alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl or heterocyclyl; wherein R³ and R⁶ may be independently optionally substituted on carbon by one or more R⁸; and wherein if said heterocyclyl contains an —NH— moiety that nitrogen may be optionally substituted by a group selected from R¹³;

R⁴, R⁵, R⁷ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;

R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

R¹² is hydroxy, methyl, ethyl, propyl or trifluoromethyl;

m is 0 or 1;

q is 0 or 1;

or a pharmaceutically acceptable salt thereof;

with the proviso that said compound is not 1-acetyl-3-(4-fluorobenzoyl)piperidine; 1-acetyl-3-(4-dimethylaminobenzoyl)piperidine; 1-(4-nitrobenzoyl)-3-(4-fluorobenzoyl)piperidine; 1-(4-aminobenzoyl)-3-(4-fluorobenzoyl)piperidine; 1-acetyl-3-(4-phthalimidobenzoyl)piperidine; 1-(benzoyl)-3-(4-mesylaminobenzoyl)piperidine; 1-(t-butoxycarbonyl)-3-(4-aminobenzoyl)piperidine; or 1,3-dibenzoylpiperidine.

In another aspect of the invention there is provided a compound of formula (IA): 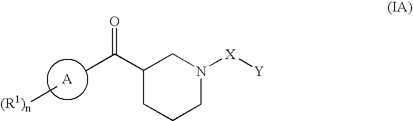
wherein:

Ring A is selected from phenyl, pyridyl, thienyl, furyl or thiazolyl;

R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl or heterocyclyl; wherein R¹ may be optionally substituted on carbon by one or more groups selected from R³; and wherein if said heterocyclyl contains an —NH— moiety that nitrogen may be optionally substituted by a group selected from R⁴;

n is 0-5; wherein the values of R¹ may be the same or different;

X is a —C(O)—, —S(O)₂—, —C(O)NR¹¹—, —C(S)NR¹¹—, —C(O)O— or —C(—NR¹¹)—; wherein R¹¹ is selected from hydrogen, C₁₋₄alkyl, carbocyclyl and heterocyclyl;

Y is C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;

R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N-(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl or heterocyclyl; wherein R² may be optionally substituted on carbon by one or more groups selected from R⁶; and wherein if said heterocyclyl contains an —NH— moiety that nitrogen may be optionally substituted by a group selected from R⁷;

R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl or heterocyclyl; wherein R³ and R⁶ may be independently optionally substituted on carbon by one or more R⁸; and wherein if said heterocyclyl contains an —NH— moiety that nitrogen may be optionally substituted by a group selected from R¹³;

R⁴, R⁵, R⁷ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

q is 0 or 1;

or a pharmaceutically acceptable salt thereof; with the proviso that said compound is not 1-acetyl-3-(4-fluorobenzoyl)piperidine; 1-acetyl-3-(4-dimethylaminobenzoyl)piperidine; 1-(4-nitrobenzoyl)-3-(4-fluorobenzoyl)piperidine; 1-(4-aminobenzoyl)-3-(4-fluorobenzoyl)piperidine; 1-acetyl-3-(4-phthalimidobenzoyl)piperidine; 1-(benzoyl)-3-(4-mesylaminobenzoyl)piperidine; 1-(t-butoxycarbonyl)-3-(4-aminobenzoyl)piperidine; or 1,3-dibenzoylpiperidine.

In this specification the term “alkyl” includes both straight and branched chain alkyl groups but references to individual alkyl groups such as “propyl” are specific for the straight chain version only. For example, “C₁₋₆alkyl” and “C₁₋₄alkyl” includes propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals therefore “carbocyclylC₁₋₄alkyl” would include 1-carbocyclylpropyl, 2-carbocyclylethyl and 3-carbocyclylbutyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

“Heteroaryl” is a totally unsaturated, mono or bicyclic ring containing 3-12 atoms of which at least one atom is chosen from nitrogen, sulphur or oxygen, which may, unless otherwise specified, be carbon or nitrogen linked. Suitably “heteroaryl” refers to a totally unsaturated, monocyclic ring containing 5 or 6 atoms or a bicyclic ring containing 8-10 atoms of which at least one atom is chosen from nitrogen, sulphur or oxygen, which may, unless otherwise specified, be carbon or nitrogen linked. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyranyl, indolyl, pyrimidyl, pyrazinyl, pyridazinyl, benzothienyl, pyridyl and quinolyl. Particularly “heteroaryl” refers to thienyl, furyl, thiazolyl, pyridyl, benzothienyl, imidazolyl or pyrazolyl.

“Aryl” is a totally unsaturated, mono or bicyclic carbon ring that contains 3-12 atoms. Suitably “aryl” is a monocyclic ring containing 5 or 6 atoms or a bicyclic ring containing 9 or 10 atoms. Suitable values for “aryl” include phenyl or naphthyl. Particularly “aryl” is phenyl.

A “heterocyclyl” is a saturated, partially saturated or unsaturated, mono, bicyclic or tricyclic ring containing 3-15 atoms of which at least one atom is chosen from nitrogen, sulphur or oxygen, which may, unless otherwise specified, be carbon or nitrogen linked, wherein a —CH₂— group can optionally be replaced by a —C(O)— or a —C(S)—, or a ring sulphur atom may be optionally oxidised to form the S-oxides. Particularly a “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂— group can optionally be replaced by a —C(O)— or a —C(S)—, or a ring sulphur atom may be optionally oxidised to form the S-oxides. More particularly a “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form the S-oxides. Preferably a “heterocyclyl” is a saturated, partially saturated or unsaturated, mono or bicyclic ring containing 5 or 6 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form S-oxide(s). Examples and suitable values of the term “heterocyclyl” are thienyl, piperidinyl, morpholinyl, furyl, thiazolyl, pyridyl, imidazolyl, 1,2,4-triazolyl, thiomorpholinyl, coumarinyl, pyrimidinyl, phthalidyl, pyrazolyl, pyrazinyl, pyridazinyl, benzothienyl, benzimidazolyl, tetrahydrofuryl, [1,2,4]triazolo[4,3-a]pyrimidinyl, piperidinyl, indolyl, 1,3-benzodioxolyl and pyrrolidinyl. Further examples and suitable values of the term “heterocyclyl” are 1,3-benzodioxolyl, thienyl, furyl, thiazolyl, pyrazinyl, pyrrolyl, indolyl, quinolinyl, isoquinolinyl, pyrazolyl, isoxazolyl, benzofuranyl, 1,2,3-thiadiazolyl, 1,2,5-thiadiazolyl, pyrimidinyl, 2,1-benzisoxazolyl, 4,5,6,7-tetrahydro-2H-indazolyl, imidazo[2,1-b][1,3]thiazolyl, tetrahydrofuranyl, tetrahydropyranyl, piperidinyl, morpholinyl, 2,3-dihydro-1-benzofuryl, 2,3-dihydro-1,4-benzodioxinyl and pyridyl. Further examples and suitable values for the term “heterocyclyl” are benzofuranyl, 2,1-benzisoxazolyl, 1,3-benzodioxolyl, 1,3-benzothiazolyl, benzothienyl, 3,4-dihydro-2H-benzodioxepinyl, 2,3-dihydro-1,4-benzodioxinyl, chromanyl, 2,3-dihydrobenzofuranyl, furyl, imidazo[2,1-b][1,3]thiazolyl, indolyl, isoindolinyl, isoquinolinyl, isoxazolyl, morpholinyl, oxazolyl, piperidinyl, pyrazinyl, pyrazolyl, pyridyl, pyridazinyl, pyrimidinyl, pyrrolidinyl, pyrrolyl, quinolinyl, quinoxalinyl, tetrahydrofuryl, 4,5,6,7-tetrahydro-1-benzofuryl, 4,5,6,7-tetrahydro-2H-indazolyl, 4,5,6,7-tetrahydro-1H-indolyl, tetrahydropyranyl, 1,2,3,4-tetrahydroquinolinyl, thiazolyl, 1,2,3-thiadiazolyl, 1,2,5-thiadiazolyl or thienyl.

A “carbocyclyl” is a saturated, partially saturated or unsaturated, mono, bicyclic or tricyclic carbon ring that contains 3-15 atoms; wherein a —CH₂— group can optionally be replaced by a —C(O)—. Particularly a “carbocyclyl” is a saturated, partially saturated or unsaturated, mono or bicyclic carbon ring that contains 3-12 atoms; wherein a —CH₂— group can optionally be replaced by a —C(O)—. Preferably “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. Particularly “carbocyclyl” is cyclohexyl, phenyl, naphthyl or 2-6-dioxocyclohexyl. More particularly “carbocyclyl” is phenyl, naphthyl, cyclopropyl, cyclopentyl, cyclohexyl, 1,2,3,4-tetrahydronaphthyl or indenyl. More particularly “carbocyclyl” is naphthyl, phenyl, cyclopropyl, cyclohexyl, indenyl, 1,2,3,4-tetrahydronaphthyl, cyclopentyl or (3r)-adamantanyl.

An example of “C₁₋₄alkanoyloxy” is acetoxy. Examples of “C₁₋₄alkoxycarbonyl” include methoxycarbonyl, ethoxycarbonyl, n- and t-butoxycarbonyl. Examples of “C₁₋₄alkoxy” include methoxy, ethoxy and propoxy. Examples of “oxyC₁₋₄alkoxy” include oxymethoxy, oxyethoxy and oxypropoxy. Examples of “C₁₋₄alkanoylamino” include formamido, acetamido and propionylamino. Examples of and “C₁₋₄alkylS(O)ₐ wherein a is 0 to 2” include methylthio, ethylthio, methylsulphinyl, ethylsulphinyl, mesyl and ethylsulphonyl. Examples of and “C₁₋₄alkylsulphonyl” include mesyl and ethylsulphonyl. Examples of “C₁₋₄alkanoyl” include propionyl and acetyl. Examples of “N—(C₁₋₄alkyl)amino” include methylamino and ethylamino. Examples of “N,N—(C₁₋₄alkyl)₂amino” include di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₄alkyl)sulphamoyl” are N-(methyl)sulphamoyl and N-(ethyl)sulphamoyl. Examples of “N—(C₁₋₄alkyl)₂sulphamoyl” are N,N-(dimethyl)sulphamoyl and N-(methyl)-N-(ethyl)sulphamoyl. Examples of “N—(C₁₋₄alkyl)carbamoyl” are methylaminocarbonyl and ethylaminocarbonyl. Examples of “N,N—(C₁₋₄alkyl)₂carbamoyl” are dimethylaminocarbonyl and methylethylaminocarbonyl. Examples of “C₁₋₄alkylsulphonylamino” are mesylamino and ethylsulphonylamino. Examples of “C₀₋₄alkylene” are a direct bond, methylene and ethylene.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

All compounds of the formula (I) have chiral centres and some may have geometric isomeric centres (E- and Z-isomers), and it is to be understood that the invention encompasses all such optical, diastereoisomers and geometric isomers that possess 11βHSD1 inhibitory activity.

The invention relates to any and all tautomeric forms of the compounds of the formula (I) that possess 11βHSD1 inhibitory activity.

It is also to be understood that certain compounds of the formula (I) can exist in solvated as well as unsolvated forms such as, for example, hydrated forms. It is to be understood that the invention encompasses all such solvated forms which possess 11βHSD1 inhibitory activity.

Particular values of variable groups are as follows. Such values may be used where appropriate with any of the definitions, claims or embodiments defined hereinbefore or hereinafter.

Definitions for Ring A

a) Ring A is aryl.
b) Ring A is heteroaryl; wherein if said heteroaryl contains an —NH— moiety that nitrogen may be optionally substituted by a group selected from R⁹.
c) Ring A is aryl or heteroaryl; wherein if said heteroaryl contains an —NH— moiety that nitrogen may be optionally substituted by a group selected from R⁹.
d) Ring A is carbocyclyl.
e) Ring A is heterocyclyl; wherein if said heterocyclyl contains an —NH— moiety that nitrogen may be optionally substituted by a group selected from R⁹.
f) Ring A is phenyl.
g) Ring A is phenyl wherein the positions ortho to the (CH₂)_{q} group are unsubstituted or substituted by fluoro, preferably unsubstituted.
   Definitions for R¹
a) R¹ is selected from halo or C₁₋₄alkyl.
b) R¹ is halo.
c) R¹ is selected from fluoro, chloro, methoxy or methyl.
d) R¹ is selected from fluoro.
   Definitions for n
a) n is 0-3; wherein the values of R¹ may be the same or different.
b) n is 0-2; wherein the values of R¹ may be the same or different.
c) n is 0 or 1.
d) n is 2; wherein the values of R¹ may be the same or different.
e) n is 1.
f) n is 0.
   Combinations of A, n and R¹
a) Ring A is phenyl, n is 1 and the substituent is para to the —(CH₂)_{q}— group of formula (I).
b) Ring A, R¹ and n together form 4-fluorophenyl, 4-chlorophenyl and 4-methoxyphenyl.
c) Ring A, R¹ and n together form 4-fluorophenyl, 3-fluorophenyl or 3,4-difluorophenyl.
   Definitions for X
a) X is —C(O)— or S(O)₂—.
b) X is —C(O)—.
c) X is —S(O)₂—.
d) X is —CH₂—.
e) X is —C(O)NR¹¹—; wherein R¹¹ is selected from hydrogen.
f) X is —C(O)NR¹¹—; wherein R¹¹ is selected from C₁₋₄alkyl.
g) X is —C(O)NR¹¹—, wherein R¹¹ is selected from methyl.
h) X is —C(S)NR¹¹—; wherein R¹¹ is selected from hydrogen.
i) X is —C(S)NR¹¹—; wherein R¹¹ is selected from C₁₋₄alkyl.
j) X is —C(O)O—.
k) X is a direct bond.
l) X is —C(═NR¹¹)—; wherein R¹¹ is selected from hydrogen.
m) X is —C(═NR¹¹)—; wherein R¹¹ is selected from C₁₋₄alkyl.
n) X is a direct bond, —C(O)—, —C(O)O— or —S(O)₂—.
   Definitions for Y
a) Y is C₁₋₆alkyl; wherein Y may be optionally substituted on carbon by one or more R².
b) Y is carbocyclyl or heterocyclyl; wherein Y may be optionally substituted on carbon by one or more R²; wherein if said heterocyclyl contains an —NH— moiety that nitrogen may be optionally substituted by a group selected from R⁵.
c) Y is carbocyclyl; wherein Y may be optionally substituted on carbon by one or more R².
d) Y is heterocyclyl; wherein Y may be optionally substituted on carbon by one or more R²; wherein if said heterocyclyl contains an —NH— moiety that nitrogen may be optionally substituted by a group selected from R⁵.
e) Y is hydrogen, C₁₋₆alkyl, carbocyclyl or heterocyclyl; wherein Y may be optionally substituted on carbon by one or more R²; wherein if said heterocyclyl contains an —NH— moiety that nitrogen may be optionally substituted by a group selected from R⁵.
f) Y is hydrogen, phenyl, thienyl, isopropyl, methyl, t-butyl, furyl, cyclopropyl, cyclohexyl, quinolinyl or benzothienyl, 1,2,5-thiadiazolyl, morpholino, pyridyl, tetrahydrofuryl or indolyl; wherein Y may be optionally substituted on carbon by one or more R².
g) Y is hydrogen, phenyl, thien-2-yl, isopropyl, methyl, t-butyl, fur-2-yl, cyclopropyl, cyclohexyl, quinolin-2-yl, quinolin-3-yl, benzothienyl, 1,2,5-thiadiazol-3-yl, morpholino, pyrid-2-yl, tetrahydrofur-2-yl or indol-6-yl; wherein Y may be optionally substituted on carbon by one or more R².
Definitions for R²
a) R² is a substituent on carbon and is selected from halo, cyano, C₁₋₄alkyl, C₁₋₄alkoxy, N—(C₁₋₄alkyl)amino or carbocyclyl; wherein R² may be optionally substituted on carbon by one or more groups selected from R⁶; wherein R⁶ is halo.
b) R² is a substituent on carbon and is selected from fluoro, chloro, cyano, trifluoromethyl, methoxy, ethoxy, isopropoxy, difluoromethoxy, trifluoromethoxy, 4-fluorophenyl, methylamino or t-butoxy.
c) When Y is phenyl, R² is para to X.
   Combinations of X and Y

X and Y together form hydrogen, t-butoxycarbonyl, cyclopropylcarbonyl, cyclohexylcarbonyl, 4-fluorobenzoyl, 2,5-difluorobenzoyl, 2-chlorobenzoyl, 2-cyanobenzoyl, 4-cyanobenzoyl, 4-methoxybenzoyl, 4-ethoxybenzoyl, 4-isopropoxybenzoyl, 4-t-butoxybenzoyl, 4-difluoromethoxybenzoyl, 2-trifluoromethoxybenzoyl, 3-trifluoromethoxybenzoyl, 4-trifluoromethoxybenzoyl, 4-methylaminobenzoyl, 4-fluorobenzylcarbonyl, thien-2-ylcarbonyl, 5-chlorothien-2-ylcarbonyl, fur-2-ylcarbonyl, 5-trifluoromethylfur-2-ylcarbonyl, morpholinocarbonyl, 1,2,5-thiadiazol-3-ylcarbonyl, quinolin-2-ylcarbonyl, quinolin-3-ylcarbonyl, pyrid-2-ylcarbonyl, tetrahydrofur-2-ylcarbonyl, indol-6-ylcarbonyl, benzothien-2-ylcarbonyl, isopropylsulphonyl, 4-fluorophenylsulphonyl, 2-trifluoromethylphenylsulphonyl or thien-2-ylsulphonyl.

Definitions of R¹²
a) R¹² is hydroxy, methyl, ethyl, propyl or trifluoromethyl;
b) R¹² is hydroxy, methyl, ethyl or propyl;
c) R¹² is hydroxy, methyl, ethyl or trifluoromethyl;
d) R¹² is methyl or ethyl.
e) R¹² is methyl.
   Definitions of m
a) m is 0.
b) m is 1.
   Definitions of q
a) q is 0.
b) q is 1.
c)
   Substituents on R¹, R², R³ and R⁴
   In one aspect R¹ is optionally substituted by 1, 2 or 3 groups selected from R³.
   In one aspect R¹ is optionally substituted by 1 or 2 groups selected from R³.
   In one aspect R¹ is optionally substituted by 1 group selected from R³.
   In one aspect R² is optionally substituted by 1, 2 or 3 groups selected from R⁶.
   In one aspect R² is optionally substituted by 1 or 2 groups selected from R⁶.
   In one aspect R² is optionally substituted by 1 group selected from R⁶.
   In one aspect R³ is optionally substituted by 1, 2 or 3 groups selected from R⁸.
   In one aspect R³ is optionally substituted by 1 or 2 groups selected from R⁸.
   In one aspect R³ is optionally substituted by 1 group selected from R⁸.
   In one aspect R⁶ is optionally substituted by 1, 2 or 3 groups selected from R⁸.
   In one aspect R⁶ is optionally substituted by 1 or 2 groups selected from R⁸.
   In one aspect R⁶ is optionally substituted by 1 group selected from R⁸.

Particular classes of compounds that are useful in the manufacture of a medicament for use in the inhibition of 11βHSD1 are disclosed in Table A using combinations of the definitions described hereinabove, wherein any other variables required in the definition are as given in the first definition of the compound of formula (I) at the beginning of the description. For example, ‘a’ in the column headed R² in the table refers to definition (a) given for R² hereinabove and ‘I’ refers to the first definition given for the variables in the compound of formula (I) at the beginning of the description. Certain classes of compounds may be novel as compounds in their own right.

TABLE A

| Class | Ring A | R¹ | n | q | X | Y | R¹² | XXm |
|---|---|---|---|---|---|---|---|---|
| 1 | I | I | a | I | I | I | I | I |
| 2 | I | I | a | I | I | I | a | a |
| 3 | c | I | b | I | I | I | a | b |
| 4 | c | a | b | I | I | a | b | a |
| 5 | d | a | c | I | a | a | c | b |
| 6 | e | I | b | I | a | e | c | b |
| 7 | f | a | c | b | b | e | c | b |
| 8 | f | b | e | a | b | f | d | b |
| 9 | g | c | f | a | c | f | d | b |
| 10 | g | d | e | a | b | g | e | b |

According to a further feature of the invention there is provided the use of a compound

Ring A is phenyl;

R¹ is halo;

n is 0-2; wherein the values of R¹ may be the same or different;

X is a direct bond, —C(O)—, —C(O)O— or —S(O)₂—;

Y is hydrogen, phenyl, thienyl, isopropyl, methyl, t-butyl, furyl, cyclopropyl, cyclohexyl, quinolinyl or benzothienyl, 1,2,5-thiadiazolyl, morpholino, pyridyl, tetrahydrofuryl or indolyl; wherein Y may be optionally substituted on carbon by one or more R²;

R² is a substituent on carbon and is selected from halo, cyano, C₁₋₄alkyl, C₁₋₄alkoxy, N—(C₁₋₄alkyl)amino or carbocyclyl; wherein R² may be optionally substituted on carbon by one or more groups selected from R⁶; wherein R⁶ is halo;

m is 0; and

q is 0;

in the manufacture of a medicament for use in the inhibition of 11βHSD1.

In another aspect of the invention, suitable compounds of the invention are any one of the Examples or a pharmaceutically acceptable salt thereof.

Another aspect of the present invention provides a process for preparing a compound of formula (I) or a pharmaceutically acceptable salt thereof which process (wherein variable groups are, unless otherwise specified, as defined in formula (I)) comprises of:
Process 1) for compounds of formula (I) wherein X is —C(O)—; reacting an amine of formula (II): 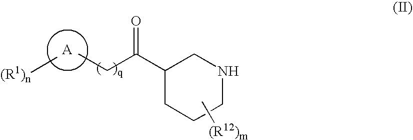
with an acid of formula (III): 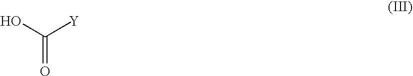
or an activated derivative thereof;
Process 2) for compounds of formula (I) wherein X is —S(O)₂—; reacting an amine of formula (II) with a sulphonyl halide of formula (IV): 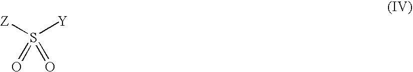
wherein Z is fluoro or chloro;
Process 3) for compounds of formula (I) wherein X is —CH₂—; reacting an amine of formula (II) with a compound of formula (V): 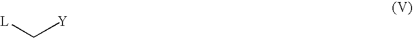
wherein L is a displaceable group;
Process 4) for compounds of formula (I) wherein X is —CH₂—; reducing a compound of formula (I) wherein X is —C(O)—;
Process 5) for compounds of formula (I) wherein X is a direct bond; reacting an amine of formula (II) with a compound of formula (VI):
L-Y (VI)
wherein L is a displaceable group;
Process 6) for compounds of formula (I) wherein X is —C(O)NR¹¹— and R¹¹ is hydrogen; reacting an amine of formula (II) with an isocyanate of formula (VII):
O═C═N—Y (VII)
Process 7) for compounds of formula (I) wherein X is —C(S)NR¹¹— and R¹¹ is hydrogen; reacting an amine of formula (II) with an isothiocyanate of formula (VIII):
S═C═N—Y (VIII)
Process 8) for compounds of formula (I) wherein X is —C(O)O—; reacting an amine of formula (II) with a compound of formula (IX):
L-C(O)—O—Y (IX)
wherein L is a displaceable group;
Process 9) for compounds of formula (I) wherein q is 0; reacting a Weinreb amide of the formula (X): 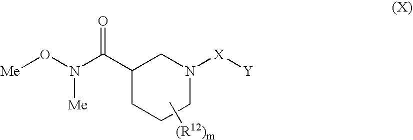
with a compound of formula (XI): 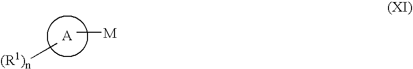
wherein M is an organometallic reagent;
Process 10) decarboxylating a compound of formula (XII): 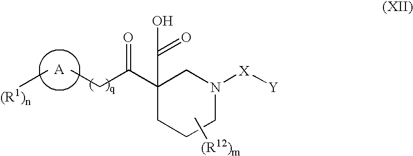
Process 11) reacting a compound of formula (XIII): 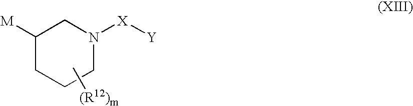
wherein M is an organometallic reagent, with a compound of formula (XIV): 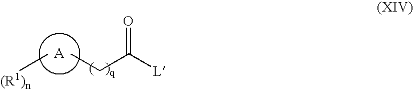
Process 12) oxidising a compound of formula (XV): 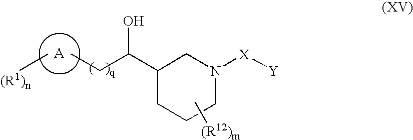
and thereafter if necessary or desirable:
i) converting a compound of the formula (I) into another compound of the formula (I);
ii) removing any protecting groups;
iii) resolving enantiomers;
iv) forming a pharmaceutically acceptable salt thereof.

An example of an activated derivative of a compound of formula (III) is the corresponding acid chloride.

L is a displaceable group, suitable values for L include halo, particularly chloro or bromo, or mesyloxy.

M is an organometallic reagent, preferably a Grignard reagent, more preferably magnesium bromide.

L′ is a leaving group, for example, halo or an activated ester.

Suitable oxidizing agents for oxidizing the hydroxyl group in a compound of the formula (XV) include Dess-Martin periodinane (1,1,1-tris(acetyloxy)-1,1-dihydro-1,2-benziodoxol-3-(1H)-one); pyridinium chlorochromate in DCM; sodium dichromate, sulfuric acid, acetone (Jones Oxidation); sodium or potassium permanganate; DMSO, oxalyl chloride, triethylamine (Swern oxidation); and hydrogen peroxide.

The reactions described above may be performed under standard conditions known to the person skilled in the art. The intermediates described above are commercially available, are known in the art or may be prepared by known procedures.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess 11βHSD1 inhibitory activity. These properties may be assessed using the following assay.

Assay

HeLa cells (human cervical carcinoma derived cells) were stably transfected with a construct containing four copies of the glucocorticoid response element (GRE) linked to a beta-galactosidase reporter gene (3 kb lac Z gene derived from pSV-B-galactosidase). These cells were then further stably transfected with a construct containing fill-length human 11βHSD1 enzyme (in pCMVHyg) to create GRE4-βGal/11βHSD1 cells. The principal of the assay is as follows. Cortisone is freely taken up by the cells and is converted to cortisol by 11βHSD1 oxo-reductase activity and cortisol (but not cortisone) binds to and activates the glucocorticoid receptor. Activated glucocorticoid receptor then binds to the GRE and initiates transcription and translation of β-galactosidase. Enzyme activity can then be assayed with high sensitivity by colourimetric assay. Inhibitors of 11βHSD1 will reduce the conversion of cortisone to cortisol and hence decrease the production of β-galactosidase.

Cells were routinely cultured in DMEM (Invitrogen, Paisley, Renfrewshire, UK) containing 10% foetal calf serum (LabTech), 1% glutamine (Invitrogen), 1% penicillin & streptomycin (Invitrogen), 0.5 mg/ml G418 (Invitrogen) & 0.5 mg/ml hygromycin (Boehringer). Assay media was phenol red free-DMEM containing 1% glutamine, 1% penicillin & streptomycin.

Compounds (1 mM) to be tested were dissolved in dimethyl sulphoxide (DMSO) and serially diluted into assay media containing 10% DMSO. Diluted compounds were then plated into transparent flat-bottomed 384 well plates (Matrix, Hudson N.H., USA).

The assay was carried out in 384 well microtitre plate (Matrix) in a total volume of 50 μl assay media consisting of cortisone (Sigma, Poole, Dorset, UK, 1 μM), HeLa GRE4-βGal/11HSD1 cells (10,000 cells) plus test compounds (3000 to 0.01 nM). The plates were then incubated in 5% O₂, 95% CO₂ at 37° C. overnight.

The following day plates were assayed by measurement of β-galactosidase production.

A cocktail (25 μl) consisting of 10× Z-buffer (600 mM Na₂HPO₄, 400 mM NaH₂PO₄.2H₂O, 100 mM KCl, 10 mM MgSO₄.7H₂O, 500 mM, β-mercaptoethanol, pH 7.0), SDS (0.2%), chlorophenol red-β-D-galactopyranoside (5 mM, Roche Diagnostics) was added per well and plates incubated at 37° C. for 34 hours. β-Galactosidase activity was indicated by a yellow to red colour change (absorbance at 570 nm) measured using a Tecan Spectrafluor Ultra.

The calculation of median inhibitory concentration (IC₅₀) values for the inhibitors was performed using Origin 6.0 (Microcal Software, Northampton Mass. USA). Dose response curves for each inhibitor were plotted as OD units at each inhibitor concentration with relation to a maximum signal (cortisone, no compound) and IC₅₀ values calculated. Compounds of the present invention typically show an IC₅₀<10 μM. For example the following results were obtained:

| Example | IC₅₀ (nm) |
|---|---|
| 13 | 10 |
| 17 | 83 |
| 18 | 206 |

According to a further aspect of the invention there is provided a pharmaceutical composition which comprises a compound of formula (IA′) or a pharmaceutically acceptable salt thereof or of the Examples, or a pharmaceutically acceptable salt thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I), or a pharmaceutically acceptable salt thereof, will normally be administered to a warm-blooded animal at a unit dose within the range 0.1-50 mg/kg that normally provides a therapeutically-effective dose. A unit dose form such as a tablet or capsule will usually contain, for example 1-1000 mg of active ingredient. However the daily dose will necessarily be varied depending upon the host treated, the particular route of administration, and the severity of the illness being treated. Accordingly the optimum dosage may be determined by the practitioner who is treating any particular patient.

We have found that the compounds defined in the present invention, or a pharmaceutically acceptable salt thereof, are effective 11βHSD1 inhibitors, and accordingly have value in the treatment of disease states associated with metabolic syndrome.

It is to be understood that where the term “metabolic syndrome” is used herein, this relates to metabolic syndrome as defined in 1) and/or 2) or any other recognised definition of this syndrome. Synonyms for “metabolic syndrome” used in the art include Reaven's Syndrome, Insulin Resistance Syndrome and Syndrome X. It is to be understood that where the term “metabolic syndrome” is used herein it also refers to Reaven's Syndrome, Insulin Resistance Syndrome and Syndrome X.

Also provided is a compound of the formula (I) as hereinabove defined for use in inhibiting 11βHSD1.

Also provided is a compound of the formula (I) as hereinabove defined for use in treating disease states associated with metabolic syndrome.

Also provided is a compound of the formula (I) as hereinabove defined for use in treating diabetes.

According to a further aspect of the present invention there is provided a compound of formula (IA′) or a pharmaceutically acceptable salt thereof or of the Examples, or a pharmaceutically acceptable salt thereof, as defined hereinbefore for use in a method of prophylactic or therapeutic treatment of a warm-blooded animal, such as man.

Thus according to this aspect of the invention there is provided a compound of formula (IA′) or a pharmaceutically acceptable salt thereof or of the Examples, or a pharmaceutically acceptable salt thereof, as defined hereinbefore for use as a medicament.

According to another feature of the invention there is provided the use of a compound of the formula of formula (IA′) or a pharmaceutically acceptable salt thereof or of the Examples, or a pharmaceutically acceptable salt thereof, as defined hereinbefore in the manufacture of a medicament for use in the production of an 11βHSD1 inhibitory effect in a warm-blooded animal, such as man.

Where production of or producing an 11βHSD1 inhibitory effect is referred to suitably this refers to the treatment of metabolic syndrome. Alternatively, where production of an 11βHSD1 inhibitory effect is referred to this refers to the treatment of diabetes, obesity, hyperlipidaemia, hyperglycaemia, hyperinsulinemia or hypertension, particularly diabetes and obesity. Alternatively, where production of an 11αHSD1 inhibitory effect is referred to this refers to the treatment of glaucoma, osteoporosis, tuberculosis, dementia, cognitive disorders or depression.

According to a further feature of this aspect of the invention there is provided a method for producing an 11βHSD1 inhibitory effect in a warm-blooded animal, such as man, in need of such treatment which comprises administering to said animal an effective amount of a compound of formula (I), or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound of formula (IA′) or a pharmaceutically acceptable salt thereof or of the Examples, or a pharmaceutically acceptable salt thereof.

In addition to their use in therapeutic medicine, the compounds of formula (I), or a pharmaceutically acceptable salt thereof, are also useful as pharmacological tools in the development and standardisation of in vitro and in vivo test systems for the evaluation of the effects of inhibitors of 11βHSD1 in laboratory animals such as cats, dogs, rabbits, monkeys, rats and mice, as part of the search for new therapeutic agents.

The inhibition of 11βHSD1 described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example agents than might be co-administered with 11βHSD1 inhibitors, particularly those of the present invention, may include the following main categories of treatment:
1) Insulin and insulin analogues;
2) Insulin secretagogues including sulphonylureas (for example glibenclamide, glipizide), prandial glucose regulators (for example repaglinide, nateglinide), glucagon-like peptide 1 agonist (GLP1 agonist) (for example exenatide, liraglutide) and dipeptidyl peptidase IV inhibitors (DPP-IV inhibitors);
3) Insulin sensitising agents including PPARγ agonists (for example pioglitazone and rosiglitazone);
4) Agents that suppress hepatic glucose output (for example metformin);
5) Agents designed to reduce the absorption of glucose from the intestine (for example acarbose);
6) Agents designed to treat the complications of prolonged hyperglycaemia; e.g. aldose reductase inhibitors
7) Other anti-diabetic agents including phosotyrosine phosphatase inhibitors, glucose 6-phosphatase inhibitors, glucagon receptor antagonists, glucokinase activators, glycogen phosphorylase inhibitors, fructose 1,6 bisphosphastase inhibitors, glutamine:fructose-6-phosphate amidotransferase inhibitors
8) Anti-obesity agents (for example sibutramine and orlistat);
9) Anti-dyslipidaernia agents such as, HMG-CoA reductase inhibitors (statins, eg pravastatin); PPARα agonists (fibrates, eg gemfibrozil); bile acid sequestrants (cholestyramine); cholesterol absorption inhibitors (plant stanols, synthetic inhibitors); ileal bile acid absorption inhibitors (IBATi), cholesterol ester transfer protein inhibitors and nicotinic acid and analogues (niacin and slow release formulations);
10) Antihypertensive agents such as, β blockers (eg atenolol, inderal); ACE inhibitors (eg lisinopril); calcium antagonists (eg. nifedipine); angiotensin receptor antagonists (eg candesartan), α antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin; and
12) Anti-inflammatory agents, such as non-steroidal anti-infammatory drugs (eg. aspirin) and steroidal anti-inflammatory agents (eg. cortisone).

In the above other pharmaceutical composition, process, method, use and medicament manufacture features, the alternative and preferred embodiments of the compounds of the invention described herein also apply.

### EXAMPLES

The invention will now be illustrated in the following Examples, in which standard techniques known to the skilled chemist and techniques analogous to those described in these Examples may be used where appropriate, and in which, unless otherwise stated:

(i) evaporations were carried out by rotary evaporation in vacuo and work up procedures were carried out after removal of residual solids such as drying agents by filtration;

(ii) all reactions were carried out under an inert atmosphere at ambient temperature, typically in the range 18-25° C., with solvents of HPLC grade under anhydrous conditions, unless otherwise stated;

(iii) column chromatography (by the flash procedure) was performed on Silica gel 40-63 μm (Merck);

(iv) yields are given for illustration only and are not necessarily the maximum attainable;

(v) the structures of the end products of the formula (I) were generally confirmed by nuclear (generally proton) magnetic resonance (NMR) and mass spectral techniques; magnetic resonance chemical shift values were measured in deuterated CDCl₃ (unless otherwise stated) on the delta scale (ppm downfield from tetramethylsilane); proton data is quoted unless otherwise stated; spectra were recorded on a Varian Mercury-300 MHz, Varian Unity plus-400 MHz, Varian Unity plus-600 MHz or on Varian Inova-500 MHz spectrometer unless otherwise stated data was recorded at 400 MHz; and peak multiplicities are shown as follows: s, singlet; d, doublet; dd, double doublet; t, triplet; tt, triple triplet; q, quartet; tq, triple quartet; m, multiplet; br, broad; ABq, AB quartet; ABd, AB doublet, ABdd, AB doublet of doublets; dABq, doublet of AB quartets; LCMS were recorded on a Waters ZMD, LC column xTerra MS C₈ (Waters), detection with a HP 1100 MS-detector diode array equipped; mass spectra (MS) (loop) were recorded on VG Platform II (Fisons Instruments) with a HP-1100 MS-detector diode array equipped; unless otherwise stated the mass ion quoted is (MH⁺);

(vi) intermediates were not generally fully characterised and purity was assessed by thin layer chromatography (TLC), HPLC, infra-red (IR), MS or NMR analysis;

(vii) where solutions were dried magnesium sulphate was the drying agent;

(viii) the following abbreviations may be used hereinbefore or hereinafter:—

DCM dichloromethane;

THF tetrahydrofuran;

HATU O-(7-azabenzotriazol-1-yl)-n,n,n′,n′-tetramethyluronium hexafluoro-phosphate;

PS-DIEA Polymer Supported-Diisopropylethylamine (From Argonaut Technologies);

DMAP 4-dimethylaminopyridine;

DMF N,N-dimethylformamide;

EDAC 1-ethyl-3-(3-dimethylaminopropyl)carbodiimide hydrochloride;

DIEA Diisopropylethylamine; and

EtOAc ethyl acetate;

ix) where an Isolute SCX-2 column is referred to, this means an “ion exchange” extraction cartridge for adsorption of basic compounds, i.e. a polypropylene tube containing a benzenesulphonic acid based strong cation exchange sorbent, used according to the manufacturers instructions obtained from International Sorbent Technologies Limited, Dyffryn Business Park, Hengeod, Mid Glamorgan, UK, CF82 7RJ;

x) where an Isolute-NH2 column is referred to, this means an “ion exchange” extraction cartridge for adsorption of acidic compounds, i.e. a polypropylene tube containing a amino silane covalently bonded to a silica particle used according to the manufacturers instructions obtained from International Sorbent Technologies Limited, Dyffryn Business Park, Hengeod, Mid Glamorgan, UK, CF82 7RJ;

xi) where as Isco CombiFlash Optix-10 parallel flash chromatography system is referred to this means an automated chromatography workstation capable of carrying out up to 10 purifications in parallel via flash chromatography using pre packed silica cartridges;

xii) where a “Biotage 90 g silica column” is referred to this means an automated chromatography workstation capable of carrying out up to 4 purifications in parallel via flash chromatography using pre packed silica cartridges, eg Si 12+M available from Biotage Inc. A Dyax Corp. Company; and

xiii) where a “Genevac HT4” is referred to, this means a centrifugal evaporator capable of the simultaneous evaporation of multiple samples supplied by Genevac Ltd, The Sovereign Centre, Farthing Road, Ipswich, Suffolk IP1 5AP, UK.

### Example 1

### (RS)1-(4-Fluorobenzoyl)-3-(4-fluorobenzoyl)piperidine

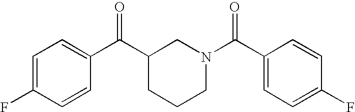

To a stirred solution of (RS)4-fluorophenyl-(3-piperidyl)methanone hydrochloride (WO 88/02365; 122 mg, 0.5 mmol) and triethylamine (210 μl, 1.5 mmol) in DCM (5 ml) was added 4-fluorobenzoyl chloride (68 mg, 0.43 mmol). The reaction was left to stir at room temperature overnight then washed with 2M HCl (2×3 ml), sat NaHCO₃ (3 ml) and brine (3 ml). The resulting solution was dried, filtered and evaporated to yield product as a solid (75 mg, 53%). M/z: 330 [M+H]⁺.

### Examples 2-9

The procedure described in Example 1 was repeated using the appropriate reagent to replace the “4-fluorobenzoyl chloride” to obtain the compounds described below.

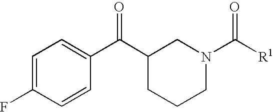

| Ex | R¹ | NMR | M/z |
|---|---|---|---|
| 2 | thien-2-yl | | 318 |
| 3 | Cyclopropyl | 0.70 (m, 2H), 0.75 (m, 2H), 1.60 (m, 1H), 1.70 (m, 1H), | 276 |
| | | 1.75 (m, 1H), 1.90 (m, 1H), 2.00 (m, 1H), 3.10 (br m, | |
| | | 2H), 3.5 (m, 1H), 4.15 (d, 1H), 4.30 (d, 1H), 7.30 (t, | |
| | | 2H), 8.05 (m, 2H) | |
| 4 | fur-2-yl | | 302 |
| 5* | morpholino | | 321 |
| 6 | 2-chlorophenyl | 1.64-2.01 (m, 3H), 2.01-2.21 (m, 1H), 2.72-3.39 (m, | 347 |
| | | 2H), 3.39-3.69 (m, 2H), 4.71-5.02 (m, 1H), 6.99-7.48 | |
| | | (m, 6H), 7.73-7.48 (m, 1H), 7.99-8.11 (m, 1H) | |
| 7 | 3- | 1.53-1.99 (br m, 3H), 2.02-2.26 (br m, 1H), 2.80-3.99 | 397 |
| | trifluoromethoxy | (br m, 4H), 4.46 -4.97 (br m, 1H), 6.97-7.55 (br m, 6H), | |
| | phenyl | 7.70-8.20 (br m, 2H) ** | |
| 8 | 4-difluoromethoxy | | 379 |
| | phenyl | | |
| 9 | 4-isopropoxy | 1.30-1.42 (d, 6H), 1.46-1.72 (br m, 3H), 1.72-1.93 (br | 371 |
| | phenyl | m, 1H), 2.01-2.15 (br m, 1H), 2.82-3.27 (br m, 2H), | |
| | | 3.27-3.62 (br m, 1H), 3.62-4.94 (br m, 1H), 4.49-4.66 | |
| | | (m, 1H), 6.81-6.93 (m, 2H), 7.04-7.21 (m, 2H), 7.32- | |
| | | 7.47 (m, 2H), 7.81-8.19 (br m, 2H) ** | |
| 10 | quinoline-2-yl | 1.57-2.03 (br m, 3H), 2.03-2.23 (m, 1H), 2.87-2.87 (m, | 364 |
| | | 2H), 3.34-3.88 (d of m, 1H), 4.09-4.25 (m, 1H), 4.73- | |
| | | 5.04 (d of m, 1H), 6.92-7.02 (m, 1H), 7.12-7.23 (m, | |
| | | 11H), 7.56-7.67 (m, 1H), 7.67-7.81 (m, 2H), 7.81-7.90 | |
| | | (m, 1H), 7.90-8.00 (m, 1H), 8.00-8.17 (m, 2H), 8.20- | |
| | | 8.32 (m, 1H) ** | |

* 4-morpholino carbamoyl chloride used in place of acid chloride.
** Significant broadening of peaks was observed.

### Examples 11-14

The procedure described in Example 1 was repeated using the appropriate sulphonyl chloride to replace the “4-fluorobenzoyl chloride” to obtain the compounds described below.

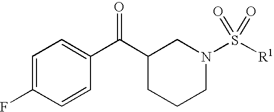

| Ex | R¹ | NMR | M/z |
|---|---|---|---|
| 11 | 4-fluorophenyl | | 366 |
| 12 | thien-2-yl | | 354 |
| 13 | isopropyl | | 314 |
| 14 | 2-trifluoromethyl | (DMSO-d₆): 1.55 (m, 1H), 1.70 (m, 1H), 1.80 (m, 1H), | 416 |
| | phenyl | 2.00 (m, 1H), 2.90 (m, 1H), 3.00 (t, 1H), 3.60 (m, 1H), | |
| | | 3.70 (br d, 1H), 3.80 (m, 1H), 7.30 (t, 2H), 7.85 (m, | |
| | | 2H), 8.00 (M, 3H), 8.10 (m, 1H) | |

### Examples 15-47

Examples 15-47 were prepared by the following general procedure.

To a solution of the appropriate carboxylic acid component (0.5 mmol) in DMF (1 ml) was added sequentially a solution of HATU (209 mgs) in DMF (1 ml), PS-DIEA (273 mgs of 3.66 mmol/g) and a sonicated solution of the appropriate aryl (piperidin-3-yl)methanone hydrochloride (see above and Methods 2 and 3; 139 mgs, 0.57 mmol) and DIEA (50.5 mgs, 0.07 ml, 0.5 mmol) in DMF (1-2 ml), and the reaction mixture aged for approximately 16 hours.

The products were purified by purification technique (a) or (b) described below:

a) The reaction mixture was poured onto an Isolute SCX-2 column (1 g, 0.4 mmol/g) aligned over an Isolute-NH2 column (1 g, 0.6 mmol/g) transferring with DCM (0.5 ml). The columns were then eluted under atmospheric pressure with DCM. The bulk of the solvent was removed using a Genevac HT4 and then if necessary purified using the Isco CombiFlash Optix-10 parallel flash chromatography Optics-10 system (12 g silica column, Gradient of isohexane/EtOAc, Flow rate 30 ml/min).

b) The bulk of the solvent was removed using a Genevac HT4 and then purified using the Isco CombiFlash Optix-10 parallel flash chromatography system Optics-10 system (12 g silica column, Gradient of isohexane/EtOAc, Flow rate 30 ml/min).

It will be appreciated that various orders of addition, and various purification methods, or combinations of methods, can be employed to prepare the compounds exemplified below, and their congeners.

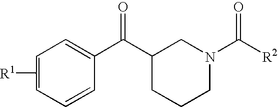

| Ex | R¹ | R² | M/z |
|---|---|---|---|
| 15 | 4-F | 1,2,5-thiadiazol-3-yl | 320 |
| 16 | 4-F | Cyclohexyl | 318 |
| 17 | 4-F | 4-fluorobenzyl | 344 |
| 18 | 4-F | 5-chlorothien-2-yl | 352 |
| 19 | 4-F | 4-cyanophenyl | 337 |
| 20 | 4-F | 4-methoxyphenyl | 343 |
| 21 | 4-F | 2,5-difluorophenyl | 349 |
| 22 | 4-F | quinolin-3-yl | 364 |
| 23 | 4-F | tetrahydrofuran-2-yl (RS) | 307 |
| 24 | 4-F | indol-6-yl | 352 |
| 25 | 4-F | benzothien-2-yl | 369 |
| 26 | 4-F | 2-trifluoromethoxyphenyl | 397 |
| 27 | 4-F | 4-ethoxyphenyl | 357 |
| 28 | 4-F | 5-trifluormethylfur-2-yl | 371 |
| 29 | 3-F | 4-trifluoromethoxyphenyl | 397 |
| 30 | 3-F | 2-cyanophenyl | 360 |
| 31 | 3-F | benzothien-2-yl | 368 |
| 32 | 3-F | 2,5-difluorophenyl | 349 |
| 33 | 3,4-di-F | 4-t-butyloxyphenyl | 403 |
| 34 | 3,4-di-F | 4-trifluoromethoxyphenyl | 414 |
| 35 | 3,4-di-F | 4-methylaminophenyl | 382** |
| 36 | 3,4-di-F | 2-cyanophenyl | 377** |
| 37 | 3,4-di-F | 4-ethoxyphenyl | 375 |
| 38 | 3,4-di-F | 2,5-difluorophenyl | 366 |
| 39 | 3,4-di-F | tetrahydrofuran-2-yl (RS) | 324*** |

| Ex | R¹ | R² | NMR | M/z |
|---|---|---|---|---|
| 40 | 4-F | pyrid-2-yl | DMSO-d₆: 1.53-1.87 (br m, 3H), 1.91-2.07 (br | 314 |
| | | | m, 1H), 2.84-3.24 (br m, 2H), 3.57-3.88 (br m, | |
| | | | 2H), 4.29-4.60 (br d of m, 1H), 7.25-7.51 (br | |
| | | | m, 3H), 7.51-7.6 (br m, 1H), 7.82-8.03 (br m, | |
| | | | 2H), 8.04-8.17 (br m, 1H), 8.52-8.62 (br m, | |
| | | | 1H) | |
| 41 | 4-F | 2-cyanophenyl | DMSO-d₆: 1.52-1.87 (br m, 3H), 1.93-2.10 (br | 338 |
| | | | m, 1H), 2.95-3.49 (br ms, 3H), 3.49-3.54 (br m, | |
| | | | 1H), 4.26-4.61 (d of m, 1H), 7.22-7.35 (m, | |
| | | | 1H), 7.35-7.44 (m, 1H), 7.51-7.69 (m, 2H), | |
| | | | 7.69-7.85 (m, 1H), 7.85-8.00 (m, 2H), 8.02- | |
| | | | 8.13 (m, 1H) | |
| 42 | 3-F | 4-t-butyloxyphenyl | DMSO-d₆+CD₃CO₂D at 100° C.: | 385 |
| | | | 1.31 (br s, 9H), 1.49-1.81 (br m, 3H), 1.89-2.07 | |
| | | | (br m, 1H), 2.92-3.45 (br m, 2H (+H₂O)), 3.52- | |
| | | | 3.70 (br m, 1H), 3.73-4.29, (br m, 2H), 6.83- | |
| | | | 7.04 (app d, 2H), 7.27-7.38 (app d, 2H), 7.38- | |
| | | | 7.49 (m, 1H), 7.49-7.62 (m, 1H), 7.62-7.72 (m, | |
| | | | 1H), 7.72-7.82 (m, 1H) | |
| 43 | 3-F | 2-trifluoromethoxy | 1.60-2.00 (br m, 3H), 2.00-2.27 (br m, 1H), | 397 |
| | | phenyl | 2.68-3.38 (br m, 2H), 3.39-3.67 (br m, 2H), | |
| | | | 4.66-5.06 (br m, 1H), 7.15-7.89 (br m, 8H) | |
| 44 | 3-F | 4-ethoxyphenyl | DMSO-d₆+CD₃CO₂D at 100° C.: 1.26-1.39 (br | 357 |
| | | | m, 3H), 1.51-1.81 (br m, 3H), 1.91-2.07 (br m, | |
| | | | 1H), 2.29-3.27 (br m, 2H), 3.52-3.68 (br m, | |
| | | | 1H), 3.80-4.00 (br m, 1H), 4.00-4.24 (br m, | |
| | | | 3H), 6.86-6.99 (br m, 2H), 7.29-7.39 (br m, | |
| | | | 2H), 7.39-7.50 (br m, 1H), 7.50-7.61 (br m, | |
| | | | 1H), 7.61-7.73 (br m, 1H), 7.73-7.82 (br m, | |
| | | | 1H) | |
| 45 | 3,4 | benzothien-2-yl | DMSO-d₆+CD₃CO₂D at 100° C.: 1.59-1.87 (br | 387 |
| | di-F | | m, 3H), 1.94-2.11 (br m, 1H), 3.14-3.40 (br m, | |
| | | | 2H), 3.62-3.75 (br m, 1H), 4.07-4.19 (br m, | |
| | | | 1H), 4.24-4.37 (br m, 1H), 7.36-7.58 (br m, | |
| | | | 3H), 7.67 (app s, 1H) 7.80-7.92 (br m, 2H), | |
| | | | 7.92-8.02 (br m, 2H) | |
| 46 | 3,4 | 2-trifluoromethoxy | 1.63-2.01 (br m, 3H), 2.01-2.20 (br m, 1H), | 414 |
| | di-F | phenyl | 2.71-3.37 (br m, 2H), 3.37-3.63 (br m, 2H), | |
| | | | 4.68-4.97 (br m, 1H), 7.11-7.53 (br m, 5H), | |
| | | | 7.55-7.74 (br m, 1H), 7.74-7.91 (br m, 1H) | |
| 47 | 3,4 | 4-methoxyphenyl | DMSO-d₆+CD₃CO₂D at 100 ° C.: 1.50-1.81 (br | 361 |
| | di-F | | m, 3H), 1.90-2.06 (br m, 1H), 2.94-3.21 (br m, | |
| | | | 2H), 3.52-3.67 (br m, 1H), 3.77 (s, 3H), 3.83- | |
| | | | 4.21 (br m, 2H), 6.87-7.02 (app d, 2H), 7.29- | |
| | | | 7.42 (app d, 2H), 7.50-7.59 (m, 1H), 7.75- | |
| | | | 7.87(br m, 1H), 7.87-7.99 (m, 1H) | |

**Corresponds to (M+Na)⁺
***Two peaks were observed of equal mass - diastereoisomers.

### Example 48

### (RS)1-tert-Butyloxycarbonyl-3-(3-fluorobenzoyl)piperidine

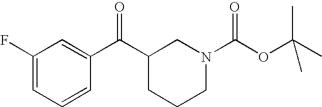

To a solution of (RS)1-tert-butyloxycarbonyl-3-(N-methyl-N-methoxycarbamoyl)piperidine (Method 1; 2.0 g, 7.3 mmol) in anhydrous THF (20 ml) at −78° C. was added 3-fluorophenylmagnesium bromide (0.5M solution in THF) at such a rate so as to maintain an internal temperature of <−60° C. On completion of addition the reaction mixture was allowed to come to room temperature and stirred for a further 16 hours. The reaction was quenched with saturated ammonium chloride solution (50 ml) and extracted with EtOAc (2×50 ml). The organics were combined, washed with water (50 ml) saturated sodium hydrogen carbonate (50 ml) and brine (50 ml), dried and evaporated in vacuo to yield 2.95 g of the title compound. NMR (DMSO-d₆): 1.34 (s, 9H), 1.42-1.64 (br m, 3H), 1.65-1.75 (br m, 1H), 1.82-1.94 (br m, 1H), 2.78-2.97 (br t, 1H), 3.43-3.57 (br s, 1H), 3.63-3.82 (br m, 1H), 3.85-4.04 (br m, 1H), 7.43-7.67 (m, 2H), 7.71 (d, 1H), 7.81 (d, 1H).

### Example 49

### (RS)1-tert-Butyloxycarbonyl-3-(3,4-difluorobenzoyl)piperidine

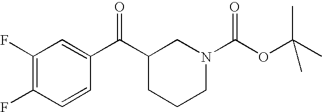

The title compound was synthesised in a manner analogous to that used in Example 48, starting from (RS)1-tert-butyloxycarbonyl-3-(N-methyl-N-methoxycarbamoyl)piperidine (Method 1) and substituting 3,4-difluorophenylmagnesium bromide (1M in THF) as the Grignard reagent. NMR (DMSO-d₆): 1.34 (app s, 10H), 1.45-1.61 (br m, 2H), 1.64-1.74 (br m, 1H), 1.82-1.95 (br m, 1H), 2.86 (br t, 1H), 3.44-3.57 (br m, 1H), 3.64-3.83 (br s, 1H), 2.84-4.00 (br s, 1H), 7.60 (q, 1H), 7.82-7.91 (m, 1H), 7.99 (app t, 1H).

### Example 50

### (RS)3-(4-Fluorobenzoyl)-1-(tert-butyloxycarbonyl)piperidine

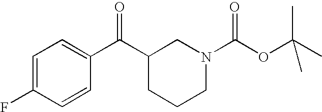

A solution of (RS)1-tert-butyloxycarbonyl-3-(N-methyl-N-methoxycarbamoyl)piperidine (Method 1; 5.0 g, 18.4 mmol) in dry THF (100 ml) was cooled (ice-bath) under argon, and a solution of 4-fluorophenyl magnesium bromide in ether (32 ml of 2M, 3.5 eq) was added dropwise with stirring, keeping the internal temperature <5° C. The reaction mixture was stirred for 16 hours, warming to ambient temperature. The reaction mixture was quenched with saturated aqueous ammonium chloride and extracted with EtOAc (3×30 ml); the extracts were combined, washed sequentially with water (2×50 ml) and brine (2×50 ml), dried and evaporated to give the crude product as a colourless oil. This was chromatographed (Biotage 90 g silica column, eluting with hexane containing EtOAc (10% v/v) to give the title compound as a colourless pasty solid (5.75 g). A sample was recrystallized from ethanol to give the title compound as colourless crystals, NMR (DMSO-d₆): 1.30 (s, 9H), 1.4-1.6 (m, 2H), 1.6-1.8 (m, 1H), 1.8-2.0 (s, 1H), 2.75-2.95 (m, 1H), 3.0-3.3 (m, 1H, signal partially obscured by HOD signal), 3.4-3.6 (m, 1H), 3.6-3.8 (m, 1H), 3.8-4.0 (s, 1H), 7.40 (t, 2H), 8.05 (m, 2H); m/z 306 [M−H]⁻, 308 [M+H]⁺.

### Examples 51-52

### (R) and (S)1-(Cyclohexylcarbonyl)-3-(4-fluorobenzoyl)piperidine

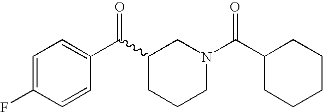

Chiral semi-preparative HPLC was used to separate the enantiomers of (RS)1-cyclohexylcarbonyl-3-(4-fluorobenzoyl)piperidine (Example 16) to give Isomers A and B, absolute stereochemistry unknown.

Conditions:

| | |
|---|---|
| Instrument | Gilson (200 ml heads) |
| Column | Merck 20 μm 50 mm Chiralpak AD No. AD00SC- |
| | HL001 |
| Eluent | EtOH |
| Oven Temperature | Ambient |
| Flow | 35 ml/min |
| Wavelength | 254 nm |
| Sample Conc. | 6.853 mg/ml in EtOH |
| Sample Volume | 10 mls (68.53 mgs) |
| Run Time | 35 mins |

| | Retention | | |
|---|---|---|---|
| | Time | | |
| Ex | (mins) | NMR | M/z |
| 51 | 12.25 | DMSO-d₆: 1.06-1.43(m, 5H), 1.43-1.83(m, 8H), 1.90-2.04(m, | 318 |
| | Isomer A | 1H), 2.45-2.65(m, 1H), 2.65-2.76(m, 1H), 3.00-3.11(m, | |
| | | 0.5H), 3.23-3.35(m, 0.5H), 3.35-3.48(m, 1H), 3.86-4.02(m, | |
| | | 1H), 4.14-4.24(m, 1H), 4.41-4.50(m, 1H), 7.34-7.42(m, 2H), | |
| | | 8.03-8.12(m, 2H) | |
| 52 | 24.44 | DMSO-d₆: 1.09-1.44(m, 5H), 1.44-1.83(m, 8H), 1.91-2.03(m, | 318 |
| | Isomer A | 1H), 2.45-2.64(m, 1H), 2.65-2.75(m, 1H), 3.01-3.11(m, | |
| | | 0.5H), 3.25-3.34(m, 0.5H), 3.35-3.60(m, 1H+H₂O), | |
| | | 3.87-4.01(m, 1H), 4.14-4.24(m, 0.5H), 4.41-4.50(m, 0.5H), (m, 1H), | |
| | | 7.33-7.41(m, 2H), 8.04-8.12(m, 2H) | |

### Example 53

### cis-1-(4-Fluorobenzoyl)-2-methyl-3-(4-fluorobenzoyl)piperidine

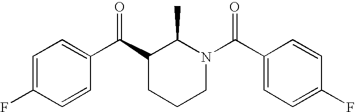

A stirred solution of cis-1-(4-fluorobenzoyl)-N-methoxy-N,2-dimethylpiperidine-3-carboxamide (Method 4; 0.2 g; 0.65 mmol; 1.0 eq.) in anhydrous tetrahydrofuran (6 ml) was cooled to 0° C. and a 2M solution of 4-fluorophenyl magnesium bromide in diethyl ether (1.135 ml; 2.27 mmol; 3.5 eq.) was added dropwise. The reaction mixture was stirred at 0° C. for 45 mins and then at ambient temperature for 5 hrs. The reaction was quenched with a saturated aqueous solution of ammonium chloride and extracted into ethyl acetate. The organic layer was washed sequentially with water and brine, then dried and concentrated in vacuo. The residue thus obtained was purified by column chromatography on silica eluting with 10-40% ethyl acetate in iso-hexanes, to give the title compound as a white solid (167 mg; 75%); NMR (DMSOd₆) 0.98 (d, 3H), 1.58 (m, 1H), 1.73 (m, 2H), 2.0 (m, 1H), 3.05 (t, 1H), 3.75 (m, 1H), 3.9 (m, 1H), 4.73 (m, 1H), 7.25 (m, 4H), 7.5 (d, 2H), 7.91 (d, 2H); m/z 344 [M+H⁺]

### Example 54

### cis-1-(4-Fluorobenzoyl)-2-methyl-3-(4-methoxybenzoyl)piperidine

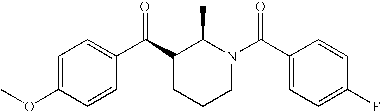 The title compound was prepared in a manner analogous to that described for Example 53, using the appropriate Grignard reagent: NMR: (DMSOd₆; 393K) 0.95 (d, 3H), 1.58 (m, 1H), 1.74 (m, 2H), 2.05 (q, 1H), 3.02 (t, 1H), 3.67 (m, 1H), 3.9 (m, 4H), 4.70 (m, 1H), 7.00 (d, 2H), 7.25 (m, 2H), 7.50 (m, 2H), 7.80 (d, 2H); m/z 356 [M+H⁺].
Preparation of Starting Materials
Method 1

### (RS)1-tert-Butyloxycarbonyl-3-(N-methyl-N-methoxycarbamoyl)piperidine

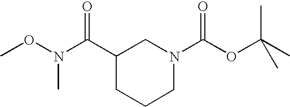

(RS)1-tert-butyloxycarbonyl-3-piperidine carboxylic acid (Murata, Toshiki; Shimada, Mitsuyuki; Kadono, Hiroshi; Sakakibara, Sachiko; Yoshino, Takashi; Masuda, Tsutomu; Shimazaki, Makoto; Shintani, Takuya; Fuchikami, Kinji; Bacon, Kevin B.; Ziegelbauer, Karl B.; Lowinger, Timothy B. Synthesis and structure-activity relationships of novel IKK-β inhibitors. Part 2: Improvement of in vitro activity. Bioorganic & Medicinal Chemistry Letters (2004), 14(15), 4013-4017; 10.0 g, 36.7 mmol) was stirred with EDAC (8.45 g, 44.1 mmol) and DMAP (13.45 g, 110.1 mmol) in DMF (50 ml) under argon for 5 minutes. N,O-dimethylhydroxylamine hydrochloride (4.30 g, 44.1 mmol) was added as a solid in one portion and the reaction was allowed to stir for 16 hours. The reaction mixture was partitioned between EtOAc (2×150 ml) and water (100 ml). The organics were combined and washed sequentially with water (100 ml), 1M citric acid (2×100 ml), saturated sodium hydrogen carbonate solution (2×100 ml) and brine (3×100 ml). The resulting solution was dried and evaporated in vacuo to give the title compound as a colourless oil (10.76 g). NMR (DMSO-d₆): 1.38 (app s, 10H), 1.47-1.56 (br d, 1H), 1.65 (d of t, 1H), 1.73-1.87 (br d, 1H), 2.64-2.84 (br m, 3H), 3.08 (s, 3H), 3.68 (s, 3H), 3.77-3.96 (m, 2H).

Method 2

### (RS)3-(3-Fluorobenzoyl)piperidine hydrochloride

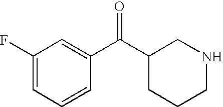

To a solution of (RS)1-tert-butyloxycarbonyl-3-(3-fluorobenzoyl)piperidine (Example 48; 2.95 g crude weight) in DCM (30 ml) was added a saturated solution of HCl in EtOAc (30 ml) with stirring. Agitation was continued for a further 4 hours, during which time a white precipitate was formed. The solvents were removed in vacuo and the residue triturated with diethyl ether (2×30 ml) to afford the title compound as a colourless solid (1.53 g). NMR (DMSO-ds): 1.43-1.63 (m, 1H), 1.69-1.89 (m, 2H), 1.94-2.06 (br d, 1H), 2.81-2.94 (br t, 1H), 3.03 (t, 1H), 3.16-3.39 (m, 2H under water), 3.77-3.92 (m, 1H), 7.49-7.67 (m, 2H), 7.26 (d of t, 1H), 7.81 (d, 1H), 8.78-8.94 (br s, 2H).

Method 3

### (RS)3-(3,4-Difluorobenzoyl)piperidine hydrochloride

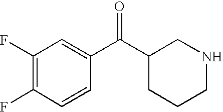

The title compound was synthesised in a manner analogous to that used in Method 2, starting from (RS)1-tert-butyloxycarbonyl-3-(3,4-difluorobenzoyl)piperidine (Example 49). NMR (DMSO-d₆): 1.51 (q of d, 1H), 1.69-2.05 (m, 3H), 2.84 (br t, 1H), 2.96 (br t, 1H), 3.22 (d, 1H), 0.3.32-3.36 (br s, 1H under water), 3.86-4.00 (m, 1H), 7.63 (q, 1H), 7.84-7.92 (m, 1H), 7.96-8.07 (m, 1H), 9.07-9.45 (br s, 2H).

Method 4

### cis-1-(4-fluorobenzoyl)-N-methoxy-N,2-dimethylpiperidine-3-carboxamide

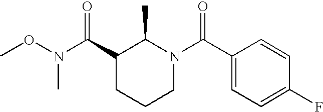

A 5M aqueous solution of sodium hydroxide (1.28 ml; 6.42 mmol; 1.0 eq.) was added to a solution of cis-ethyl 1-(4-fluorobenzoyl)-2-methylpiperidine-3-carboxylate (1.88 g; 6.42 mmol; 1.0 eq.) in methanol (6 ml). The solution was stirred at ambient temperature for 3 hours then concentrated to give 1-(4-fluorobenzoyl)-2-methylpiperidine-3-carboxylic acid as a crude oil, which was used without purification or characterisation. N,O-dimethyl hydroxylamine (0.689 g; 7.06 mmol; 1.1 eq.), N-methylmorpholine (0.78 ml; 7.06 mmol; 1.1 eq.) and 1-ethyl-3-(3-dimethylaminopropyl)carbodiimide hydrochloride (1.35 g; 7.06 mmol; 1.1 eq.) were added to a solution of the crude 1-(4-fluorobenzoyl)-2-methylpiperidine-3-carboxylic acid (1.70 g; 6.42 mmol; 1.0 eq.) in anhydrous dichloromethane (35 ml). The reaction mixture was stirred at ambient temperature for 40 hours, then washed sequentially with a 1M solution of hydrochloric acid and a saturated solution of sodium bicarbonate. The organic layer was separated and dried over anhydrous magnesium sulphate, filtered and concentrated in vacuo to give the title compound as a yellow oil (1.29 g; 65%); NMR: 1.19 (m, 3H), 1.63 (m, 5H), 2.13 (m, 1H), 2.89(m, 1H), 3.14 (m, 4H), 3.40-3.81 (m, 3H), 7.09 (m, 2H), 7.39 (m, 2H); m/z 309.5 [M+H⁺].

The requisite cis-ethyl 1-(4-fluorobenzoyl)-2-methylpiperidine-3-carboxylate was prepared as follows: 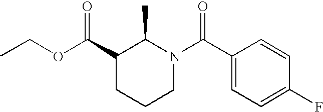
4-Fluorobenzoyl chloride (2.07 ml; 17.5 mmol; 1.1 eq.) and triethylamine (4.44 ml; 31.9 mmol; 2.0 eq.) were added to a solution of cis-ethyl 2-methylpiperidine-3-carboxylate (CAS 110287-64-4; U.S. Pat. No. 5,494,919A; 2.73 g; 16.0 mmol; 1.0 eq.) in anhydrous dichloromethane (55 ml). The suspension was stirred at ambient temperature and under an inert atmosphere for 72 hours. The solvent was removed under reduced pressure and the residue dissolved in ethyl acetate and washed sequentially with water, saturated sodium bicarbonate, 1M aqueous citric acid, water and brine. The organic layer was separated, dried over anhydrous magnesium sulphate, filtered and concentrated in vacuo. The residue was purified by column chromatography on silica eluting with 30-60% ethyl acetate in iso-hexanes, to give the cis-ethyl 1-(4-fluorobenzoyl)-2-methylpiperidine-3-carboxylate (2.45 g; 52%); NMR: 1.18 (m, 3H), 1.26 (t, 3H), 1.46-1.67 (m, 2H), 1.93 (m, 2H), 2.70-3.55 (m, 2H), 4.13 (q, 2H), 4.25-5.45 (m,2H), 7.09 (m, 2H), 7.39 (m, 2H); m/z 294 [M+H⁺].

1. The use of a compound of formula (I): 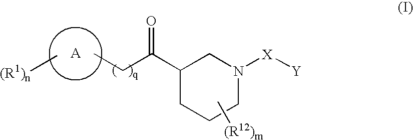
wherein:
Ring A is selected from carbocyclyl or heterocyclyl; wherein if said heterocyclyl contains an —NH— moiety that nitrogen may be optionally substituted by a group selected from R⁹;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;
n is 0-5; wherein the values of R¹ may be the same or different;
X is a direct bond, —C(O)—, —S(O)₂—, —C(O)NR¹¹—, —C(S)NR¹¹—, —C(O)O—, —C(═NR¹¹)— or —CH₂—; wherein R¹¹ is selected from hydrogen, C₁₋₄alkyl, carbocyclyl and heterocyclyl;
Y is hydrogen, C₁₋₆alkyl, C₂₋₆alkenyl, C₂₋₆alkyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸; and wherein if said heterocyclyl contains an —NH— moiety that nitrogen may be optionally substituted by a group selected from R¹³;
R⁴, R⁵, R⁷, R⁹ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is hydroxy, methyl, ethyl, propyl or trifluoromethyl;
m is 0 or 1;
q is 0 or 1;
or a pharmaceutically acceptable salt thereof;
in the manufacture of a medicament for use in the inhibition of 11βHSD1.

2. The use of a compound according to claim 1, wherein ring A is aryl or heteroaryl; wherein if the heteroaryl contains an —NH— moiety that nitrogen may be optionally substituted by a group selected from R⁹ as defined in claim 1.

3. The use of a compound according to either claim 1 or claim 2 wherein R¹ is selected from halo or C₁₋₄alkyl.

4. The use of a compound according to any one of claims 1 to 3 wherein n is 0, 1, 2 or 3.

5. The use of a compound according to any one of claims 1 to 4 wherein X is —(O)— or —S(O)₂—.

6. The use of a compound according to any one of claims 1 to 5 wherein Y is carbocyclyl or heterocyclyl; wherein Y may be optionally substituted on carbon by one or more R² as defined in claim 1 and wherein if said heterocyclyl contains an —NH— moiety that nitrogen may be optionally substituted by a group selected from R⁵ as defined in claim 1.

7. The use of a compound according to any one of claims 1 to 5 wherein Y is hydrogen, phenyl, thienyl, isopropyl, methyl, t-butyl, furyl, cyclopropyl, cyclohexyl, quinolinyl, benzothienyl, 1,2,5-thiadiazolyl, morpholino, pyridyl, tetrahydrofuryl or indolyl; wherein Y may be optionally substituted on carbon by one or more R² as defined in claim 1.

8. The use of a compound according to any one of claims 1 to 7 wherein R² is selected from halo, cyano, C₁₋₄alkyl, C₁₋₄alkoxy, N—(C₁₋₄alkyl)amino or carbocyclyl; wherein R² may be optionally substituted on carbon by one or more halo groups.

9. The use of a compound according to any one of claims 1 to 4 wherein X and Y together form hydrogen, t-butoxycarbonyl, cyclopropylcarbonyl, cyclohexylcarbonyl, 4-fluorobenzoyl, 2,5-difluorobenzoyl, 2-chlorobenzoyl, 2-cyanobenzoyl, 4-cyanobenzoyl, 4-methoxybenzoyl, 4-ethoxybenzoyl, 4-isopropoxybenzoyl, 4-t-butoxybenzoyl, 4-difluoromethoxybenzoyl, 2-trifluoromethoxybenzoyl, 3-trifluoromethoxybenzoyl, 4-trifluoromethoxybenzoyl, 4-methylaminobenzoyl, 4-fluorobenzylcarbonyl, thien-2-ylcarbonyl, 5-chlorothien-2-ylcarbonyl, fur-2-ylcarbonyl, 5-trifluoromethylfur-2-ylcarbonyl, morpholinocarbonyl, 1,2,5-thiadiazol-3-ylcarbonyl, quinolin-2-ylcarbonyl, quinolin-3-ylcarbonyl, pyrid-2-ylcarbonyl, tetrahydrofur-2-ylcarbonyl, indol-6-ylcarbonyl, benzothien-2-ylcarbonyl, isopropylsulphonyl, 4-fluorophenylsulphonyl, 2-trifluoromethylphenylsulphonyl or thien-2-ylsulphonyl.

10. The use of a compound according to any one of claims 1 to 9 wherein R¹² is hydroxy, methyl, ethyl or trifluoromethyl.

11. The use of a compound according to any one of claims 1 to 10 wherein m is 1.

12. The use of a compound according to any one of claims 1 to 11 wherein q is 0.

13. A compound of formula (IA′): 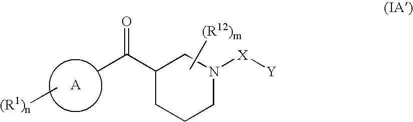
wherein:
Ring A is selected from phenyl, pyridyl, thienyl, furyl or thiazolyl;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl or heterocyclyl; wherein R¹ may be optionally substituted on carbon by one or more groups selected from R³; and wherein if said heterocyclyl contains an —NH— moiety that nitrogen may be optionally substituted by a group selected from R⁴;
n is 0-5; wherein the values of R¹ may be the same or different;
X is a —C(O)—, —S(O)₂—, —C(O)NR¹¹—, —C(S)NR¹¹—, —C(O)O— or —C(═NR¹¹)—; wherein R¹¹ is selected from hydrogen, C₁₋₄alkyl, carbocyclyl and heterocyclyl;
Y is C₁₋₆alkyl, C₂₋₆alkenyl, C₂₋₄alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl or heterocyclyl; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl or heterocyclyl; wherein R³ and R⁶ may be independently optionally substituted on carbon by one or more R⁸; and wherein if said heterocyclyl contains an —NH— moiety that nitrogen may be optionally substituted by a group selected from R¹³;
R⁴, R⁵, R⁷ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
R¹² is hydroxy, methyl, ethyl, propyl or trifluoromethyl;
m is 0 or 1;
q is 0 or 1;
or a pharmaceutically acceptable salt thereof;
with the proviso that said compound is not 1-acetyl-3-(4-fluorobenzoyl)piperidine; 1-acetyl-3-(4-dimethylaminobenzoyl)piperidine; 1-(4-nitrobenzoyl)-3-(4-fluorobenzoyl)piperidine; 1-(4-aminobenzoyl)-3-(4-fluorobenzoyl)piperidine; 1-acetyl-3-(4-phthalimidobenzoyl)piperidine; 1-(benzoyl)-3-(4-mesylaminobenzoyl)piperidine; 1-(t-butoxycarbonyl)-3-(4-aminobenzoyl)piperidine; or 1,3-dibenzoylpiperidine.

14. A compound according to claim 13 wherein R¹ is selected from halo or C₁₋₄alkyl.

15. A compound according to either claim 13 or 14 wherein n is 0, 1, 2 or 3.

16. A compound according to any one of claims 13 to 15 wherein X is —C(O)— or —S(O)₂—.

17. A compound according to any one of claims 13 to 16 wherein Y is carbocyclyl or heterocyclyl; wherein Y may be optionally substituted on carbon by one or more R² as defined in claim 1 and wherein if said heterocyclyl contains an —NH— moiety that nitrogen may be optionally substituted by a group selected from R⁵ as defined in claim 1.

18. A compound according to any one of claims 13 to 17 wherein Y is phenyl, thienyl, isopropyl, t-butyl, furyl, cyclopropyl, cyclohexyl, quinolinyl or benzothienyl; wherein Y may be optionally substituted on carbon by one or more R² as defined in claim 1.

19. A compound according to any one of claims 13 to 18 wherein R² is a substituent on carbon and is selected from halo, cyano, C₁₋₄alkyl or C₁₋₄alkoxy; wherein R² may be optionally substituted on carbon by one or more halo groups.

20. A compound according to any one of claims 13 to 19 wherein X and Y together form t-butoxycarbonyl, cyclopropylcarbonyl, cyclohexylcarbonyl, benzoyl, 4-fluorobenzoyl, 2,5-difluorobenzoyl, 2-chlorobenzoyl, 4-chlorobenzoyl, 2-cyanobenzoyl, 4-ethoxybenzoyl, 4-isopropoxybenzoyl, 4-difluoromethoxybenzoyl, 2-trifluoromethoxybenzoyl, 3-trifluoromethoxybenzoyl, thien-2-ylcarbonyl, 5-trifluoromethylfur-2-ylcarbonyl, quinoline-2-ylcarbonyl, benzothien-2-ylcarbonyl, isopropylsulphonyl, 4-fluorophenylsulphonyl or thien-2-ylsulphonyl.

21. A compound according to any one of claims 13 to 20 wherein R¹² is hydroxy, methyl, ethyl or trifluoromethyl.

22. A compound according to any one of claims 13 to 21 wherein m is 1.

23. A compound of the formula a) as defined in claim 1 selected from:
(RS)-1-(4-fluorobenzoyl)-3-(4-fluorobenzoyl)piperidine;
(RS)-1-(2-thienylcarbonyl)-3-(4-fluorobenzoyl)piperidine;
(RS)-1-cyclopropylcarbonyl-3-(4-fluorobenzoyl)piperidine;
(RS)-1-(2-furylcarbonyl)-3-(4-fluorobenzoyl)piperidine;
(RS)-1-(morpholinocarbonyl)-3-(4-fluorobenzoyl)piperidine;
(RS)-1-(2-chlorobenzoyl)-3-(4-fluorobenzoyl)piperidine;
(RS)-1-(3-trifluoromethoxybenzoyl)-3-(4-fluorobenzoyl)piperidine;
(RS)-1-(4-difluoromethoxybenzoyl)-3-(4-fluorobenzoyl)piperidine;
(RS)-1-(4-isopropoxybenzoyl)-3-(4-fluorobenzoyl)piperidine;
(RS)-1-(2-quinolincarbonyl)-3-(4-fluorobenzoyl)piperidine;
(RS)-1-(4-fluorobenzenesulphonyl)-3-(4-fluorobenzoyl)piperidine;
(RS)-1-(2-thienylsulphonyl)-3-(4-fluorobenzoyl)piperidine;
(RS)-1-isopropylsulphonyl-3-(4-fluorobenzoyl)piperidine;
(RS)-1-(2-trifluoromethylbenzenesulphonyl)-3-(4-fluorobenzoyl)piperidine;
(RS)-1-(1,2,5-thiadiazol-3-ylcarbonyl)-3-(4-fluorobenzoyl)piperidine;
(RS)-1-(cyclohexylcarbonyl)-3-(4-fluorobenzoyl)piperidine;
(RS)-1-(2-(4-fluorophenyl)acetyl)-3-(4-fluorobenzoyl)piperidine;
(RS)-1-(5-chloro-2-thienylcarbonyl)-3-(4-fluorobenzoyl)piperidine;
(RS)-1-(4-cyanobenzoyl)-3-(4-fluorobenzoyl)piperidine;
(RS)-1-(4-methoxybenzoyl)-3-(4-fluorobenzoyl)piperidine;
(RS)-1-(2,5-difluorobenzoyl)-3-(4-fluorobenzoyl)piperidine;
(RS)-1-(3-quinolincarbonyl)-3-(4-fluorobenzoyl)piperidine;
(RS)-1-(2-tetrahydrofurylcarbonyl)-3-(4-fluorobenzoyl)piperidine;
(RS)-1-(6-indolylcarbonyl)-3-(4-fluorobenzoyl)piperidine;
(RS)-1-(benzothien-2-ylcarbonyl)-3-(4-fluorobenzoyl)piperidine;
(RS)-1-(2-trifluoromethoxybenzoyl)-3-(4-fluorobenzoyl)piperidine;
(RS)-1-(4-ethoxybenzoyl)-3-(4-fluorobenzoyl)piperidine;
(RS)-1-(5-trifluoromethylfur-2-ylcarbonyl)-3-(4-fluorobenzoyl)piperidine;
(RS)-1-(4-trifluoromethoxybenzoyl)-3-(3-fluorobenzoyl)piperidine;
(RS)-1-(2-cyanobenzoyl)-3-(3-fluorobenzoyl)piperidine;
(RS)-1-(benzothien-2-ylcarbonyl)-3-(3-fluorobenzoyl)piperidine;
(RS)-1-(2,5-difluorobenzoyl)-3-(3-fluorobenzoyl)piperidine;
(RS)-1-(4-t-butoxybenzoyl)-3-(3,4-difluorobenzoyl)piperidine;
(RS)-1-(4-trifluoromethoxybenzoyl)-3-(3,4-difluorobenzoyl)piperidine;
(RS)-1-(4-methylaminobenzoyl)-3-(3,4-difluorobenzoyl)piperidine;
(RS)-1-(2-cyanobenzoyl)-3-(3,4-difluorobenzoyl)piperidine;
(RS)-1-(4-ethoxybenzoyl)-3-(3,4-difluorobenzoyl)piperidine;
(RS)-1-(2,5-difluorobenzoyl)-3-(3,4-difluorobenzoyl)piperidine;
(RS)-1-(2-tetrahydrofurylcarbonyl)-3-(3,4-difluorobenzoyl)piperidine;
(RS)-1-(2-pyridylcarbonyl)-3-(4-fluorobenzoyl)piperidine;
(RS)-1-(2-cyanobenzoyl)-3-(4-fluorobenzoyl)piperidine;
(RS)-1-(4-t-butoxybenzoyl)-3-(3-fluorobenzoyl)piperidine;
(RS)-1-(2-trifluoromethoxybenzoyl)-3-(3-fluorobenzoyl)piperidine;
(RS)-1-(4-ethoxybenzoyl)-3-(3-fluorobenzoyl)piperidine;
(RS)-1-(benzothien-2-ylcarbonyl)-3-(3,4-difluorobenzoyl)piperidine;
(RS)-1-(2-trifluoromethoxybenzoyl)-3-(3,4-difluorobenzoyl)piperidine;
(RS)-1-(4-methoxybenzoyl)-3-(3,4-difluorobenzoyl)piperidine;
(RS)-1-(t-butyloxycarbonyl)-3-(3-fluorobenzoyl)piperidine;
(RS)-1-(t-butyloxycarbonyl)-3-(3,4-difluorobenzoyl)piperidine;
(RS)-1-(t-butyloxycarbonyl)-3-(4-fluorobenzoyl)piperidine;
(R)- or (S)-1-cyclohexylcarbonyl-3-(4-fluorobenzoyl)piperidine;
(S)- or (R)-1-cyclohexylcarbonyl-3-(4-fluorobenzoyl)piperidine;
cis-1-(4-fluorobenzoyl)-2-methyl-3-(4-fluorobenzoyl)piperidine; and
cis-1-(4-fluorobenzoyl)-2-methyl-3-(4-methoxybenzoyl)piperidine;
or a pharmaceutically acceptable salt thereof.

24. A pharmaceutical composition, which comprises a compound of formula (IA′), or a pharmaceutically acceptable salt thereof, as claimed in claim 13, in association with a pharmaceutically-acceptable diluent or carrier.

25. A compound of the formula (IA′), or a pharmaceutically acceptable salt thereof, as claimed in claims 13, for use in a method of prophylactic or therapeutic treatment of a warm-blooded animal, such as man.

26. A compound of the formula (IA′), or a pharmaceutically acceptable salt thereof, as claimed in claims 13, for use as a medicament.

27. The use of a compound of the formula (I) or (IA′), or a pharmaceutically acceptable salt thereof, as claimed in claims 1 or 13, in the manufacture of a medicament for use in the production of an 11βHSD1 inhibitory effect in a warm-blooded animal, such as man.

28. The use as claimed in any one of claims 1-13 and 27 wherein production of, or producing an, 11βHSD1 inhibitory effect refers to the treatment of metabolic syndrome.

29. The use as claimed in any one of claims 1-13 and 27 wherein production of, or producing an, 11βHSD1 inhibitory effect refers to the treatment of diabetes, obesity, hyperlipidaemia, hyperglycaemia, hyperinsulinemia or hypertension, particularly diabetes and obesity.

30. The use as claimed in any one of claims 1-13 and 27 wherein production of, or producing an, 11βHSD1 inhibitory effect refers to the treatment of glaucoma, osteoporosis, tuberculosis, dementia, cognitive disorders or depression.

31. A method of producing an 11βHSD1 inhibitory effect in a warm-blooded animal, such as man, in need of such treatment which comprises administering to said animal an effective amount of a compound of formula (I), as claimed in any one of claims 1-12, or a compound of formula (IA′) as claimed in claim 13, or a pharmaceutically acceptable salt thereof.
